(19) United States
(12) Patent Application Publication
(10) Pub. No.: US 2008/0286373 A1
(43) Pub. Date: Nov. 20, 2008

(54) Ziprasidone formulations

(76) Inventors: Nageswara R. Palepu, Southampton, PA (US); Bhanu Teja Bulusu, Hyderabad (IN)

Correspondence Address: Irving M. Fishman;c/o Cohen Tauber Spievack & Wagner, Suite 2400, 420 Lexington Avenue, New York, NY, 10170, US

(21) Appl. No.: 12/152,744

(22) Filed: May 16, 2008

Related U.S. Application Data
(60) Provisional application No. 60/930,824, filed on May 18, 2007.

(51) Int. Cl. A61K 9/14; A61K 31/496; A61P 25/00

(52) U.S. Cl. 424/494; 514/254.04

(57) ABSTRACT

A ziprasidone formulation containing at least (a) one ziprasidone compound and at least an excipient component (b) that includes at least one of
(i) one or more of a mono-, di-, or tri-ester of C₁₂₋₂₄fatty acids and glycerol, in which each fatty acid group is chosen independently of the others, or mixtures thereof; and/or (ii) one or more mono- or di-esters of C₁₂₋₂₄fatty acids and polyC₂₋₃alkyleglycol, in which each fatty acid group is chosen independently of the others, or mixtures thereof; and/or (iii) a TPGS (tocopherol-succinic acid-polyethyleneglycol); and where this component (b) may optionally include (iv) optionally free polyC₂₋₃alkyleglycol; (v) optionally free glycerol; and (vi) optionally free fatty acids having 12-24 carbon atoms; and (vii) mixtures thereof;
   the formulation further comprising (c) at least one surfactant selected from anionic and non-ionionic surfactants and still further comprising (d) at least one hydroxylalkyl alkylcellulose in which each alkyl group and each hydroxyalkyl group independently has from 1 to 4 carbon atoms. The formulation achieves improved dissolution and bioavailability of the formulation. Reduction in side effect profile and increased efficacy and utility in additional indications are also disclosed.

### CROSS-REFERENCE TO RELATED APPLICATIONS

Not Applicable

### STATEMENT REGARDING FEDERALLY SPONSORED RESEARCH OR DEVELOPMENT

Not Applicable

### FIELD OF THE INVENTION

The present invention relates to the field of ziprasidone and its salts and to increasing the solubility thereof as well as enhancing the dissolution rate ziprasidone in formulations (including pharmaceutical formulations) thereof.

### BACKGROUND OF THE INVENTION

Ziprasidone (as the monohydrochloride monohydrate) is available from Pfizer under the tradename GEODON. The free base has the structure:

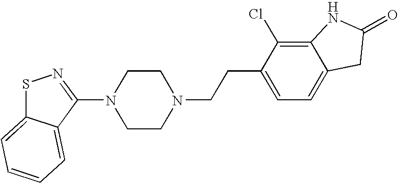

and it has a rather low solubility and is claimed in U.S. Pat. No. 4,831,031 (incorporated herein in its entirety by reference). In U.S. Pat. No. 6,150,366 (incorporated herein in its entirety by reference), limiting the mean particle size to <85 microns, preferably to <5-30 microns in a ziprasidone formulation results in a product having an AUC or Cₘₐₓ>125% that observed from an identical formulation except that the mean particle size is about 85 microns. In these tests the dissolution tests were conducted at pH 7.5 in 900 ml of aqueous monobasic sodium phosphate containing 2w/v % Na dodecyl sulfate using a USP 2 apparatus with paddles rotating at 75 rpm. Within 45 minutes, 70% of the ziprasidone was dissolved.

Other means of attempting to improve ziprasidone solubility include providing the ziprasidone as the monohydrate, hemihydrate, and anhydrate as seen in U.S. Pat. No. 5,312,925; preparing prodrugs as in U.S. Pat. No. 5,935,960; preparing ziprasidone mesylate hydrates as in U.S. Pat. No. 6,110,918 and U.S. Pat. No. 6,245,765; and preparing various inclusion complexes of ziprasidone with cyclodextrin as in U.S. Pat. No. 6,232,304 and U.S. Pat. No. 6,399,777. Unfortunately, those avenues to date having greater water solubility have significant impediments or undesirable characteristics while others may increase solubility only slightly at the expense of product stability. A recent publication, WO/2005/123086, discusses formulations of ziprasidone of mean particle size of at least 90 microns having the same or improved bioavailability as the identical formulation except that in the comparison formulation not of WO/2005/123086 the mean particle size is not greater than 85 microns. (All of the foregoing patents and patent applications are incorporated herein in their entireties by reference.)

### OBJECTS OF THE INVENTION

It is therefore an object of the invention to provide a system for increasing the aqueous solubility of ziprasidone or a salt thereof (whether anhydrates, hydrates, or other solvates thereof) thereby increasing the dissolution rate in gastric and intestinal milieu.

It is a further object of the invention to provide a formulation for ziprasidone or a salt thereof (whether anhydrates, hydrates, or other solvates thereof) which demonstrates increased aqueous solubility and enhanced dissolution over the identical formulation in the absence of the invention requirements.

It is still another object of the invention to provide a solubilization system comprising a combination of excipients for ziprasidone or a salt thereof (whether anhydrates, hydrates, or other solvates thereof) which demonstrates synergistic improvements in ziprasidone (or a pharmaceutically acceptable salt thereof) (whether anhydrates, hydrates, or other solvates thereof) aqueous solubility and enhanced dissolution over the identical formulation in the absence of one or more of the components of the synergistic system.

It is a further object of the invention to provide a method of manufacture of a ziprasidone or a salt thereof (whether anhydrates, hydrates, or other solvates thereof) formulation containing a solubilization system comprising a combination of excipients to enhance dissolution of ziprasidone (or a pharmaceutically acceptable salt thereof) (whether anhydrates, hydrates, or other solvates thereof) in water, Simulated Gastric Fluid (SGF) and/or Simulated Intestinal Fluid (SIF).

Yet another object of the invention is to provide a method of use of the formulations of the invention in the treatment of ziprasidone responsive conditions.

A still further object of the invention is to achieve therapeutic equivalency to GEODON with a formulation having less active agent than the GEODON formulation to which it is compared.

Yet another objective of the invention is to provide a once daily dosage form of ziprasidone (or a pharmaceutically acceptable salt thereof).

A still further objective of the invention is to provide a formulation of ziprasidone (or a pharmaceutically acceptable salt thereof) which formulation has a substantially similar pharmacokinetic profile and/or dissolution profile and/or absorption profile when taken with food as when taken in the absence of food.

It is further objective of this invention to provide a dosage form of ziprasidone meeting at least one of the foregoing objects said dosage form being selected from a tablet, a capsule, a dispersible tablet, an orally disintegrating tablet, a suspension for oral administration, an injectable form, or a transdermal patch.

Still another object of the invention is to provide a formulation of ziprasidone (or a pharmaceutically acceptable salt thereof) having substantially therapeutic equivalents with marketed GEODON but having a lesser amount of active agent such that a reduced level of side effects in therapy with the invention formulation is achieved as compared to the substantially therapeutically equivalent GEODON.

Still further objects of the invention will be appreciated by those of ordinary skill in the art.

### BRIEF SUMMARY OF THE INVENTION

These and other objects of the invention can be achieved by a ziprasidone formulation containing at least (a) one ziprasidone compound and at least an excipient component (b) that includes at least one of
(i) one or more of a mono-, di-, or tri-ester of C₁₂₋₂₄fatty acids and glycerol, in which each fatty acid group is chosen independently of the others, or mixtures thereof; and/or
(ii) one or more mono- or di-esters of C₁₂₋₂₄fatty acids and polyC₂₋₃alkyleglycol, in which each fatty acid group is chosen independently of the others, or mixtures thereof; and/or
(iii) a vitamin E TPGS (Vitamin E tocopherol-succinic acid-polyethyleneglycol); and where this component (b) may optionally include
(iv) optionally free polyC₂₋₃alkyleglycol;
(v) optionally free glycerol; and
(vi) optionally free fatty acids having 12-24 carbon atoms; and
(vii) mixtures thereof;
   the formulation further comprising
   (c) at least one surfactant selected from anionic and non-ionionic surfactants and still further comprising (d) at least one hydroxylalkyl alkylcellulose in which each alkyl group and each hydroxyalkyl group independently has from 1 to 4 carbon atoms.

### BRIEF DESCRIPTION OF THE SEVERAL VIEWS OF THE DRAWING

Not Applicable

### DETAILED DESCRIPTION OF THE INVENTION

The present invention is an enhanced aqueous solubility of ziprasidone (its pharmaceutically acceptable salts, whether or not hydrated or solvated) (hereinafter “ziprasidone compounds”) by including in a formulation of at least (a) one ziprasidone compound and at least an excipient component (b) that includes at least one of
(i) one or more of a mono-, di-, or tri-ester of C₁₂₋₂₄fatty acids and glycerol, in which each fatty acid group is chosen independently of the others, or mixtures thereof; and/or
(ii) one or more mono- or di-esters of C₁₂₋₂₄fatty acids and polyC₂₋₃alkyleglycol, in which each fatty acid group is chosen independently of the others, or mixtures thereof; and/or
(iii) a vitamin E TPGS (Vitamin E tocopherol-succinic acid-polyethyleneglycol); and where this component (b) may optionally include
(iv) optionally free polyC₂₋₃alkyleglycol;
(v) optionally free glycerol; and
(vi) optionally free fatty acids having 12-24 carbon atoms; and
(vii) mixtures thereof;
   the formulation further comprising
   (c) at least one surfactant selected from anionic and non-ionionic surfactants and still further comprising (d) at least one hydroxylalkyl alkylcellulose in which each alkyl group and each hydroxyalkyl group independently has from 1 to 4 carbon atoms. These formulations can further optionally contain other pharmaceutically acceptable excipients including, without limitation, binders, fillers, disintegrants (such as, without limitation, croscarmellose, crospovidone, or sodium starch glycollate), lubricants (such as, without limitation, magnesium stearate, stearic acid), processing aids (such as, without limitation, flow aids such as talc, various compression aids, such as non-hygroscopic sugars (such as, without limitation, lactose) and sugar alcohols that are known in the pharmaceutical arts as adding flow properties, or are compression aids, or processing aides (such as, without limitation, mannitol, xylitol, sorbitol)), colors, etc, and mixtures thereof, that are generally known in the pharmaceutical dosage form arts. Preferably, one embodiment of the formulation contains a mixture of components (b)(i) and (b)(ii) and optionally further contains one or more of components (b)(iv) through (b)(vii). Most preferably, this mixture of components (b)(i), (b)(ii), and optionally (b)(iv) through (b)(vii) is available commercially under the name Gelucire available from Aventis. An alternative embodiment has (b)(iii) as component (b). Still other embodiments contain (b)(iii) in conjunction with at least one component selected from (b)(i) and (b)(ii) above. A preferred embodiment contains a Gelucire mixture, a TPGS, or a blend of the two as component (b).

The ziprasidone compound is free ziprasidone base or a pharmaceutically acceptable acid addition salt thereof, and the acid addition salt may be a salt of ziprasidone with either inorganic or organic pharmaceutically acceptable acids, or mixtures thereof. The pharmaceutically acceptable organic acids are carboxylic acid or sulfonic acids, such as, without limitation, lactic acid, tartaric acid, citric acid, acetic acid, fumaric acid, malic acid, maleic acid, formic acid, oxalic acid, methanesulfonic acid, ethanesulfonic acid, benzenesulfonic acid, succinic acid, glutamic acid, and adipic acid. The pharmaceutically acceptable inorganic acid may be hydrochloric acid, hydrobromic acid, hydroiodic acid, phosphoric acid (or its mono or dibasic form), sulfuric acid (or its mono basic form bisulfate), among others known in the pharmaceutical formulation arts. In some formulations, the ziprasidone compound is a blend of such organic acid addition salt and either ziprasidone free base or ziprasidone inorganic acid addition salt, or a blend of all three, ziprasidone free base, ziprasidone organic acid addition salt and ziprasidone inorganic acid addition salt, most preferably a mixture of ziprasidone hydrochloride and ziprasidone organic acid addition salt. The blend can arise in situ by reacting ziprasidone inorganic acid addition salt with an organic acid or other manners one of ordinary skill in the art would appreciate or by physically mixing the different acid addition salts of ziprasidone and/or base.

The GELUCIRE line of compositions are inert semi-solid waxy materials which are amphiphilic in character and are available with varying physical characteristics. They are surface active in nature and disperse or solubilize in aqueous media forming micelles, microscopic globules or vesicles. They are identified by their melting point/HLB value. The melting point is expressed in degrees Celsius and the HLB (Hydrophile-Lipophile Balance) is a numerical scale extending from 0 to approximately 20. Lower HLB values denote more lipophilic and hydrophobic substances, and higher values denote more hydrophilic and lipophobic substances. The affinity of a compound for water or for oily substances is determined and its HLB value is assigned experimentally. One or a mixture of different grades of GELUCIRE excipient may be chosen to achieve the desired characteristics of melting point and/or HLB value. As to the chemistry of GELUCIRE compositions, they are polyglycolized glycerides that are prepared by the alcoholysis reaction of natural oils with polyethylene glycols (PEG). They are mixtures of monoesters, diesters and/or triesters of glycerides of long chain (C₁₂ to C₁₈) fatty acids, and PEG (mono- and/or di-) esters of long chain (C₁₂ to C₁₈) fatty acids and can include free polyethyleneglycol (PEG) and free glycerol. GELUCIRE compositions are generally described herein as fatty acid esters of glycerol and PEG esters or as polyglycolized glycerides. The large family of GELUCIRE compositions is characterized by a wide range of melting points of from about 33° C. to about 64° C. and most commonly from about 35° C. to about 55° C., and by a variety of HLB values of from about 1 to about 14, most commonly from about 7 to about 14. For example, GELUCIRE 50/13 designates a melting point of approximately 50° C. and an HLB value of about 13 while GELUCIRE 44/14 designates a melting point of approximately 44° C. and an HLB value of about 14 to this grade. The appropriate choice of melting point/HLB value of a GELUCIRE or a mixture of GELUCIRE compositions will provide the delivery characteristics needed for a specific function, e.g., immediate release, sustained release, and the like. GELUCIRE 50/13 is composed of fatty acid (majority of C₁₆ and C₁₈) esters of glycerol, PEG esters of fatty acids (majority of C₁₆ and C₁₈), and free PEG. A particularly preferred Gelucire for use in the present invention is Gelucire 44/14; another is Gelucire 50/13.

When not using Gelucire materials, the individual components thereof may be used in place thereof as long as there is at least one long chain fatty acid mono or di ester of polyalkyleneglycol present and/or at least one mono- or di-fatty acid ester of glycerol present, preferably both. Free glycerol and free polyalkylene glycol are each optionally present or absent as desired, but they are generally present as not being separated from the esterification reaction mixture in each case when the esters are made. The polyalkoxylated ester of long chain fatty acid is selected from those having polyethoxy or polypropoxy or mixed poly(ethoxy/propoxy) groups. The mixed poly(ethoxy/propoxy) can be random or block copolymers of these groups and can be as short as those 10 alkoxy units to as much as 40 alkoxy units (i.e. a molecular weight of this portion of about 840 to about 3320). In the block copolymers, the blocks can be random or structured as in the poloxamers (polypropyleneoxide block bounded by polyethyleneoxide blocks) or the reverse poloxamers (polyethyleneoxide block bounded by polypropylene blocks). Preferably, the polyalkyleneoxide is monomeric. Most preferably, the polyalkoxylated portion is a polyethoxy group and preferably is about 15 to about 20 alkoxy units thereof (i.e., a molecular weight of this portion of about 1260 to about 1660), more preferably about 17 to about 18 repeating units (i.e., about molecular weight 1500. The long chain fatty acid component is preferably a fatty acid, whether saturated, monounsaturated, diunsaturated or polyunsaturated, having at least 12 carbon atoms and up to 24 carbon atoms, and preferably includes the 12, 14, 16, 18, 20, 22, and 24 unsaturated and monounsaturated fatty acids. Individual pure compounds may be used, but more likely and preferably mixtures of the various esters within these definitions will be used, especially because many of these compounds are not economically available in pure form. For example, many fatty acids are available in inexpensive forms as blends of fatty acids, which are then esterified with a particular polyalkyleneglycol or more likely a blend of a particular polyalkyleneglycol having a range of molecular weights. Thus, the polyalkyleneglycol ester of long chain fatty acids is likely to have a range of molecular weight ranges in the polyalkylene glycol portion and a range of fatty acid components present with in a single product. An alternative to the fatty acid esters that can be used in the present invention is TPGS. This is a succinic acid which is diesterified; on one side it is esterified to tocopherol and on the other side it is esterified with polyethyleneglycol. Also acceptable are similar materials using other pharmaceutically acceptable diacids in place of the succinic acid (such as without limitation, malic acid and maleic acid) that are similarly esterified. The polyethyleneglycol group in the TPGS can be of a wide variety of sizes, but is typically polyethyleneglycol 1000. Smaller and larger sizes for this portion of the molecule as may be desired, but when using the tocopherol compounds, it is preferable to use TPGS 1000. When using TPGS or its analogs, they are used in the same range of amounts as the fatty acid diester component set forth above.

The surfactant component can be either anionic or nonionic or mixtures thereof, with anionic being more highly preferred. Suitable anionic surfactant can be any anionic surfactant and includes, without limitation, surfactants having at least one ionized (in aqueous solution) —COO⁻; —SO₃⁻; —PO₃H₂⁻, —PO₃H⁻²; —PO₃⁻³, group. Generally these have no other charged groups present. These charged groups will be pendent upon a wide variety of lipophilic portions which include alkyl, aryl, heteroaryl, non-arylcarbocyclyl, non-arylheterocyclyl, polyalkoxy, alkyl-polyalkoxy, etc. (the rings being from 5 to 7 ring members which may be unsubstituted or substituted in a variety of ways and may be fused to other rings). The anionic surfactants are typically used as the ionic salts of alkali metals, alkaline earth metals, and/or ammonium ions, with mixtures thereof being suitable as well. Preferably, the ionic surfactants are used as the sodium salts. A most highly preferred anionic surfactant is sodium lauryl sulfate. Another is sodium dioctylsulfosuccinate. Other exemplary surfactants of this type are the sulfated, or phosphorylated fatty alcohols and the corresponding fatty alcohol-PEG-sulfate or phosphorylate, where the PEG interrupting groups is of various lengths as desired. Additional anionic surfactants include fatty acids esterified to the hydroxyl end of a hydroxy acid, the carboxylic acid being in the salt form thereof. Another version is a fatty alcohol esterified to one end of a diacid (such as succinic acid, malic acid, maleic acid, etc), the other end being a free carboxy group in the salt form. Corresponding types of sulfated or phosphorylated materials are known in the art and will be apparent to those of ordinary skill from the disclosure herein. Many of these are well known in the detergent arts and are commercially available from a wide variety of sources. Each of these that is pharmaceutically acceptable is suitable for use in the present invention. In addition, when the phosphorylates are used, a single phosphate group can link two fatty alcohol groups and still have the negative charge required for an anionic surfactant as in (RO)₂P(O)—O—. The nonionic surfactants can be, without limitation, materials such as the polyethoxylene oxides, polypropylene oxides, copolymers of polyethylene oxides and polypropylene oxides (such as random copolymers thereof or block copolymers such as the poloxamers (polypropylene block flanked on each side by polyethylene glycol blocks, Lutrol F-127=poloxamer 407 is a preferred poloxamer) or the reverse poloxamers (polyethylene block flanked on each side by polypropylene blocks)), polysorbates (typically available under the name TWEEN), etc. In addition, the nonionic surfactants include materials similar to the anionic surfactants except that the oxygen bearing the charge in the anionic surfactants is esterified to an additional fatty group (i.e., that is having from 12 to 24 carbon atoms). Thus the anionic surfactant sodium laurylsulfate has dilaurylsulfate as a nonionic analog thereof and the anionic surfactants monolaurylphosphate and dilaurylphosphate have trilaurylphosphate as a nonoionic analog thereof. Again, each of these is suitable for use as a nonionic surfactant for the present invention as long as they are pharmaceutically acceptable.

The hydroxyalkyl-alkylcellulose component is a hydroxyC₁₋₃alkyl-C₁₋₃alkylcellulose and includes hydroxymethyl methylcellulose, hydroxyethyl methylcellulose, hydroxypropyl methylcellulose, hydroxymethyl ethylcellulose, hydroxyethyl ethylcellulose, hydroxypropyl ethylcellulose, hydroxymethyl propylcellulose, hydroxyethyl propylcellulose, and hydroxypropyl propylcellulose, most preferably hydroxypropyl methylcellulose. The viscosity grade of the hydroxyalkyl-alkylcellulose (measured at 2% solution in water at 25° C.) is preferably in the range of about 1 to about 50 cps, preferably less than about 10 cps, more preferably less than about 5 cps, most preferably about 3 cps. The most preferred hydroxyalkyl alkylcellulose is hydroxypropyl methyl cellulose 3 cps. These are commercially available from a wide variety of sources.

In addition, other excipients may be present, but need not be. These include, without limitation, fillers such as, without limitation, saccharides (inclusive of mono and di-saccharides and the corresponding sugar alcohols, such as lactose, mannose, glucose, mannitol, sorbitol, xylitol, etc.); binders (such as, without limitation, polyvinylpyrrolidone, hydroxypropyl cellulose, hydroxyethyl cellulose, corn starch and pregelatinized corn starch); dispersants (such as, without limitation, croscarmellose sodium, crospovidone, sodium alginate and sodium starch glycollate); lubricants (such as, without limitations, magnesium stearate, stearic acid, talc, glyceryl behanate); colorants, processing aids, coating materials, etc.

With respect to amounts of the components present, the components can be present in a wide range of ratios. In particular dosage forms, the components are:
(a) 10-120 parts by weight ziprasidone or a salt thereof;
(b) 20-240 parts by weight of (i) fatty acid mono and/or diesters of polyalkyleneglycols and/or (ii) mono, di, and/or tri esters of fatty acids and glycerin, and (iii) optionally polyalkyleneglycols and/or free glycerol;
(c) 20-360 parts by weight hydroxyalkyl-alkylcellulose; and
(d) 20-240 parts by weight anionic and/or nonionic surfactant.

In a more preferred embodiment the present invention is directed to a formulation having
about 20 parts by weight ziprasidone (or the corresponding amount of a ziprasidone salt)
about 30-50 parts by weight Gelucire 44/14
about 30-50 parts by weight sodium lauryl sulfate and
about 40-80 parts by weight hydroxypropylmethylcellulose.

In a still more highly preferred embodiment, the present invention is directed to a ziprasidone (or the corresponding amount of a ziprasidone salt) containing formulation comprising:
about 20 parts by weight ziprasidone (or the corresponding amount of a ziprasidone salt)
about 40 parts by weight Gelucire 44/14
about 40 parts by weight sodium lauryl sulfate and
about 60 parts by weight hydroxypropylmethylcellulose.

In an even more highly preferred embodiment, the present invention is directed to a ziprasidone (or the corresponding amount of a ziprasidone salt) containing formulation comprising:
about 20 parts by weight ziprasidone (or the corresponding amount of a ziprasidone salt)
about 40 parts by weight Gelucire 44/14
about 60 parts by weight PEG 6000
about 40 parts by weight sodium lauryl sulfate and
about 60 parts by weight hydroxypropylmethylcellulose.

In a most highly preferred embodiment, the present invention is directed to a ziprasidone (or the corresponding amount of a ziprasidone salt) containing formulation comprising:
about 20 parts by weight ziprasidone (or the corresponding amount of a ziprasidone salt)
about 40 parts by weight Gelucire 44/14
about 60 parts by weight PEG 6000
about 40 parts by weight sodium lauryl sulfate
about 60 parts by weight hydroxypropylmethylcellulose and
about 50 parts by weight of croscarmellose sodium.

Within the broad ranges of the foregoing amounts, each relative to the amount of ziprasidone (calculated as free base) present, the fatty acid ester component (b) is present in the range of 24:1 to 1/3:1, preferably 12:1 to 1:1, more preferably 6:1 to 1.5:1, even more preferably about 2:1 relative to ziprasidone (calculated as free ziprasidone base). The surfactant component (c), within the broad ranges previously presented is preferably present in an amount of from 24:1 to 1/3:1, preferably 12:1 to 1:1, more preferably 6:1 to 1.5:1, even more preferably about 2:1 relative to ziprasidone (calculated as free ziprasidone base). The hydroxyalkylalkylcellulose component (d), within the broad ranges previously presented is preferably present in an amount of from36:1 to 1/6:1, preferably 18:1 to 1:1, more preferably 12:1 to 1.5:1, even more preferably about 3:1 to about 2:1 relative to ziprasidone (calculated as free ziprasidone base).

The ziprasidone for use in the present invention can be prepared in any of the manners set forth in the art as evidenced in part by at least one of the patents set forth above. However, a particularly advantageous manner of making the ziprasidone is to utilize a lyophilization process to obtain non-crystalline ziprasidone compounds. A particularly useful process is disclosed in pending patent application (U.S. Ser. No. 11/282,507, filed Nov. 18, 2005, incorporated herein by reference). Other lyophilization techniques may also be used without departing from the spirit of the invention.

In formulating the invention as a typical tablet or capsule, the fatty acid ester component (b) and/or the Vitamin E TPGS component along with any auxiliary polyalkyleneglycol is warmed to melt the components. The active agent is blended with this melt, followed by the disintegrant. The mixture is allowed to cool and solidify. The mass is triturated with the surfactant component and then blended with the hydroxyalkylalkylcellulose. The other optional components can be added at any point along the way as desired. The lubricants, colors, and other auxiliary optional materials, can then be added and if desired, the granules can be filled into capsules. Alternatively, the granules can be compressed into tablets. When desired, instead of the above, the melt, containing the ziprasidone, the fatty acid ester and/or TPGS component (b) can be diluted with a solvent and either spray dried or sprayed onto inert spheres, preferably inert sugar spheres. The spray dried material or the dried, loaded sugar spheres can then be blended with the surfactant component (c) and hydroxyalkylalkyl cellulose component (d). In either case, the mixture is either filled into capsules or compressed into tablets, or may be used as a dispersible powder for reconstitution in forms such as oral suspension or powder for dissolution for oral, parenteral, or topical administration, or for inclusion into a transdermal dosage form. The spray dried material can be granulated with disintegrant and water soluble excipients and compressed into tablets such that these tablets when kept in the mouth disperses rapidly in the mouth. The rapid disintegrating tablet may contains flavors and sugars or taste masking excipients. Alternatively, the melt containing the ziprasidone, the fatty acid component and/or TPGS can be formulated either as an ointment or as a lotion or a patch to deliver the drug transdermally.

In another preferred embodiment the formulation having at least the ziprasidone compound, the fatty acid ester and/or TPGS components (b), anionic and/or nonionic surfactant (c), and the hydroxyalkylalkylcellulose component (d) is sprayed onto inert spheres, generally inert sugar spheres, to load the formulation on or in the sphere. In these embodiments, the components that are required (other than the solvents used for dissolution of the formulation) as well as the amounts of the components and the ratios between the components are as described earlier. The components are generally prepared by dissolving the hydroxyalkylalkylcellulose along with a surfactant in the solvent or solvent blend being used. Any polyalkyleneoxide (such as PEG, etc) and/or fatty acid esters in the formulation are then added to the solution, followed by the ziprasidone compound to result in a suspension or dispersion. The inert spheres are then spray coated with the suspension/dispersion. Suitable solvents for dissolving the components for these embodiments include, without limitation, chlorinated solvents, such as, without limitation chloroform, methylene chloride, etc; cyclic ethers such as, without limitation, dioxane, tetrahydrofuran, etc., and hydroxyl solvents, such as, without limitation, lower alkanols (such as methanol, ethanol, propanol, isopropanol, etc.); and mixtures thereof. Generally, a carboxylic acid is included in order to help solubilize the ziprazidone, and this may be added as a separate component or in whole or in part as a salt of the carboxylic acid and ziprasidone in place of a corresponding amount of ziprasidone. Any of the carboxylic acids mentioned above as suitable for forming salts of ziprasidone for use in the present invention may be used as the solubility aid carboxylic acid here. A particularly preferred embodiment uses lactic acid and/or ziprasidone lactate. When being used as a solubility aid, the carboxylic acids are generally used in concentrated amounts, for example, when used in this manner, lactic acid is used preferably at concentrations in water of at least 80%, more preferably at concentrations of at least 85%.

Still another preferred embodiment of the present invention is to dissolve or disperse the ziprasidone (optionally with a carboxylic acid) or a carboxylic acid salt of ziprasidone along with the surfactant component in a melt of the fatty acid ester and/or TPGS component. The melt is then extruded as spheres, which are then coated with the hydroxyalkyl-alkylcellulose component. To aid dissolution, the same carboxylic acids set forth above can be used in the same manner as set forth above.

Once formulated, the invention product can be used in any of the indications for which ziprasidone is known to be useful. In addition, due to the enhanced properties of the formulation, the present invention may be used in a manner that (a) reduces the side effect profile because lower dosages of active agent can be administered to achieve the same therapeutic effect as the already marketed GEODON; (b) achieves efficacy in patients for which efficacy could not have been achieved previously (due to the better dissolution), (c) achieves efficacy in conditions for which the currently marketed GEODON did not have sufficient efficacy in at all. Thus, the ziprasidone formulations of the present invention find application in the treatment of, without limitation, patients exhibiting the symptoms of schizophrenia, bipolar mania and depression and/or metabolic disorders, among others.

### EXAMPLES

The following non-limiting Examples are designed to exemplify, not limit, the scope of the invention, which is limited only by the claims appended hereto.

### Example 1

The solubility of ziprasidone hydrochloride monohydrate was tested in various solvents as shown in Table 1 below. Solubility in various excipients that are frequently used to improve solubility was also tested and the results thereof are in Table 2 below.

TABLE 1

| | Ziprasidone HCl |
|---|---|
| solvent | monohydrate |
| DMF/DMA/DMSO | 1.5% |
| Methanol | 1.5% |
| Acetone | <1% |
| Ethanol | <1% |
| Isopropanol | <1% |
| DCM | <1% |
| THF | <1% |
| ACN | <1% |
| DMF/water 80/20 | <1% |
| Methanol/water 60/40 | <1% |
| Methanol/water 80/20 | 1.5% |
| Acetone/water | <1% |
| Methanol/acetic acid 90/10 | <1% |

TABLE 2

| EXCIPIENT | SOLUBILITY | |
|---|---|---|
| Water | 0.03 | mg/ml |
| 0.1 N HCl solution | 0.5 | mg/ml |
| Phosphate buffer pH 6.8 (without SLS) | <0.01 | mg/ml |
| PEG 400 | 2.5 | mg/ml |
| PEG 6000 | 2.5 | mg/ml |
| Tween 80 | 2.1 | mg/ml |
| Tween 20 | 3.0 | mg/ml |
| Glycerol | 1.25 | mg/ml |
| Propylene glycol | 2 | mg/ml |
| Span 80 | 0.5 | mg/ml |
| Lactic Acid (85% strength) | 10 | mg/ml |
| Gelucire 44/14 | 1.0 | mg/ml |
| Gelucire 50/13 | 1.0 | mg/ml |
| Labrofac (Capric triglyceride PEG-4 ester) | 0.9 | mg/ml |
| 20% Lutrol in TPGS | 1.0 | mg/ml |
| Peceol (glycerol monooleate 40) | 1.0 | mg/ml |
| Maisine 35-1 (glycerol monolinoleate) | 1.0 | mg/ml |
| Ethanol | 0.4 | mg/ml |
| N-methyl 2-pyrrolidone | 10 | mg/ml |
| Benzyl alcohol | 2.9 | mg/ml |
| Benzyl benzoate | 0.3 | mg/ml |
| Glacial Acetic acid | 2.8 | mg/ml |
| 20% Lactic acid in PEG 400 | 1.3 | mg/ml |
| 5% caprolactam in solution in water | 0.3 | mg/ml |

### Example 2

Various formulations were then prepared and tested (see Table 3) for dissolution against currently marketed GEODON product formulation using USP-1 (Basket method) using 900 ml of 0.05 M monobasic sodium phosphate buffer as a medium (in which the pH is 6.8) and a volume of 900 ml. The test results are set forth in Table 3. All the inventive formulations are dissoluting a higher amount of the drug in the first one hour compared to the marketed product and the drug is precipitating out at later points due the saturation solubility in the dissolution medium. In-vivo the precipitation of the drug may not occur as the dissolved drug is constantly moving in the upper and lower GI tract and mixes with the intestinal fluids which contain bile acid and other enzymes that will keep the drug in the dissolved form. Based on these dissolution data it is anticipated that formulations presented Table 3 may show a higher bioavailability compared to the marketed product. These formulations can easily be prepared by melting the Gelucire and the PEG 6000 in a glass beaker, and ziprasidone is added while the mass is still molten, followed by the Ac-DiSol (while still molten). The mixture is allowed to solidify by keeping it at about room temperature. The mass is then triturated and the sodium lauryl sulfate (SLS) (or alternate anioinic surfactant) and the HPMC are added. The mixture is then sifted and filled into gelatin capsules.

TABLE 3

| Formulation # | Formulation | | 0.5 hr | 1 hr | 1.5 hr | 2.0 hr |
|---|---|---|---|---|---|---|
| GEODON- | Innovaotors | | 0.1 | 1.8 | 2.0 | 1.6 |
| 20 | formulation | | | | | |
| 1 | ZP | 20 | 0.5 | 5.4 | 5.2 | 4.3 |
| | GEL. 44/14 | 40 | | | | |
| | PEG 6000 | 60 | | | | |
| | SLS | 40 | | | | |
| | HPMC 3 cp | 60 | | | | |
| | Ac-Di-Sol | 30 | | | | |
| 2 | ZP | 20 | 0.6 | 4.2 | 2.9 | 2.4 |
| | GEL. 44/14 | 40 | | | | |
| | PEG 6000 | 60 | | | | |
| | Lutrol F 127 | 40 | | | | |
| | HPMC 3 cps | 60 | | | | |
| | Ac-Di-Sol | 30 | | | | |
| 3 | ZP | 20 | 4.4 | 4.4 | 4.2 | 3.9 |
| | GEL. 44/14 | 40 | | | | |
| | PEG 6000 | 60 | | | | |
| | SLS | 40 | | | | |
| | HPMC 3 cps | 60 | | | | |
| | Ac Di Sol | 50 | | | | |
| 4 | ZP | 20 | 3.0 | 4.7 | 4.7 | 3.8 |
| | VIT.E TPGS | 40 | | | | |
| | PEG 6000 | 60 | | | | |
| | SLS | 40 | | | | |
| | HPMC 3 cps | 60 | | | | |
| | Ac-Di-Sol | 50 | | | | |

### Example 3

| Ingredient | Qty (mg) |
|---|---|
| Ziprasidone HCl | 20 |
| Gelucire 44/14 | 40 |
| Tween-80 | 20 |
| Lactose | 70 |
| Ac-Di-Sol | 20 |

Procedure:

The Gelucire was melted and Tween 80 was added to the melt. The Ziprasidone HCl was then added thereto and mixed well. Half the amount of the Ac-Di-Sol (croscarmellose) was then added and mixed well, followed by adding the lactose, and mixed well. The remaining half of the Ac-Di-Sol was then added and the blend mixed well. The blend was then filled into size I hard gelatin capsules and utilized for testing as detailed below.

Dissolution Test Parameters:

| | |
|---|---|
| Medium | pH 6.8 phosphate buffer (Without SLS) |
| Volume | 900 mL |
| Method | USP I |
| RPM | 75 |
| Collection points | 30 min., 1 h, 1½ h, and 2 h |

| Time | Avg. % drug rel. |
|---|---|
| 30 min. | 1.2 |
| 1 hr | 1.1 |
| 1.5 hr | 9.5 |
| 2 hr. | 1.1 |

We observed almost 10% drug released at the end of 1.5 hours but most precipitated out after that time point. Several compositions of Ziprasidone were tested which showed the dissolution profile similar to that of the formulation presented in the example 3. The compositions of these formulations are presented below in examples 4-7.

### Example 4

| Ingredient | Qty (mg) |
|---|---|
| Ziprasidone HCl | 20 |
| Gelucire 44/14 | 40 |
| PEG 6000 | 60 |
| Sodium Lauryl Sulfate (SLS) | 40 |
| Ac-Di-Sol | 30 |
| Lactose | 60 |

Procedure:

Gelucire 44/14 and PEG 6000 were placed in a glass beaker and molten and mixed at 60° C. The ziprasidone HCl was added to the molten mass and mixed well. The Ac-di-Sol was then added and mixed well, followed by addition of the SLS with further mixing. The lactose was then added and mixed well. The blend was then filled into #1 hard gelatin capsules.

### Example 5

| Ingredient | Qty (mg) |
|---|---|
| Ziprasidone HCl | 20 |
| Gelucire 44/14 | 40 |
| PEG 6000 | 60 |
| Docusate sodium | 40 |
| Ac-Di-Sol | 30 |
| Lactose | 60 |

Procedure:

The procedure of Example 4 was followed except that docusate Sodium (aka dioctyl sodium sulfosuccinate) was used in place of the SLS.

### Example 6

| S. No. | Ingredient | Qty (mg) |
|---|---|---|
| 1 | Ziprasidone HCl | 20 |
| 2 | Gelucire 44/14 | 40 |
| 3 | PEG 6000 | 60 |
| 4 | SLS | 40 |
| 5 | HPMC 3 CPS | 60 |
| 6 | Ac-Di-Sol | 30 |

Procedure:

The procedure of Example 4 was used except that the HPMC was added with the SLS.

### Example 7

| Ingredient | Qty (mg) |
|---|---|
| Ziprasidone HCl | 20 |
| Gelucire 44/14 | 40 |
| PEG 6000 | 60 |
| SLS | 40 |
| HPMC 3 cps | 60 |
| Ac-Di-Sol | 50 |

Procedure:

Gelucire 44/14 and PEG 6000 were melted in a glass beaker. Ziprasidone HCl was added to the melt and mixed well, to which theca-Di-sol was added and mixed well. This was allowed to solidify at room temperature for about 30 minutes. This blend was then triturated and the SLS and the HMPMC were added and mixed well. The mixture was then sifted through a 30 mesh screen and the result was filled into size #0 hard gelatin capsules. Dissolution testing was carried out in pH 6.8 buffer at pH 6.8 in the presence of varying amount of sodium lauryl sulfate being added to the dissolution medium (separate from SLS in the dosage form).

| | |
|---|---|
| | Avg. % drug release in pH 6.8 dissolution |
| Time | media containing varying amount of SLS |

| (hours) | 0.05% SLS | 0.1% SLS | 0.2% SLS | 0% SLS |
|---|---|---|---|---|
| 0.5 | 5.0 | 20.0 | 18.0 | 4.3 |
| 1.0 | 20.8 | 31.9 | 56.5 | 2.8 |
| 1.5 | 25.1 | 39.0 | 68.8 | 2.2 |
| 2 | 26.8 | 43.3 | 71.9 | 2.3 |

As can be seen from the above dissolution data, the drug release is enhanced by the presence of a surfactant which is precisely what occurs in the GI tract (especially in the intestinal tract). The amount of bile acids present in the fed state is 10 mM and in the fasted state is 2 mM. The 0.05% SLS correspond to 1.74 mM solution which is similar to the fasted condition and 0.2% SLS is 7.5 mM solution which is similar to the fed condition.

### Example 8

In the next set of experiment, we removed the SLS from the formulation of example 7 and replaced Ac-di-sol with sodium starch glycollate. The composition is presented in the table below:

| Ingredient | Qty (mg) |
|---|---|
| Ziprasidone HCl | 20 |
| Gelucire 44/14 | 40 |
| PEG 6000 | 60 |
| HPMC 3 cps | 80 |
| Sodium starch glycollate | 40 |

The dissolution studies were conducted in pH 6.8 phosphate buffer in the presence of varying amount of SLS in the dissolution media. The dissolution data is presented below:

| Time | Avg. % drug release | | |
|---|---|---|---|
| (hours) | 0.05% SLS | 0.1% SLS | 0.2% SLS |
| 0.5 | 5.7 | 13.0 | 19.5 |
| 1 | 11.4 | 24.3 | 49.5 |
| 1.5 | 14.6 | 33.0 | 62.0 |
| 2 | 16.4 | 40.8 | 68.9 |

The release profile is slightly slower but not significantly different from that of the Example 7

### Example 9

An additional novel approach to formulate is by dissolving the ziprasidone in lactic acid and mixing the resultant solution with excipients.

| Ingredient | Qty (mg) | |
|---|---|---|
| Ziprasidone HCl | 20 | |
| Lactic acid (85%) | 2 | ml |

Procedure:

20 mg of ziprasidone HCl was dissolved in 2 ml of (85%) lactic acid to result in ziprasidone/lactatic acid solution (ZP lactate solution). The ZP solution was then used as set forth below.

| Ingredient | Qty (mg) |
|---|---|
| ZP lactate solution | 2 ml |
| Ziprasidone | 2 g |
| hydrochloride | |
| Aerosil-200 | 0.89 g |

Procedure:

2 ml of Ziprasidone lactate solution was placed in a beaker, to which the Aerosil −200 was added and triturated to get a free flowing powder. The powder was mixed well and and placed into size 0 hard gelatin capsules.

Dissolution Test Parameters:

| | |
|---|---|
| Medium | D.I Water |
| Volume | 900 mL |
| Method | USP I |
| RPM | 75 |
| Collection points | 30 min., 1 hr 1.30 hr, 2 hr |

| Time | Avg. % drug rel. |
|---|---|
| 30 min. | 70.8 |
| 60 min | 54.8 |
| 90 min | 77.8 |
| 120 min | 52.0 |

From the above data, it can be seen that about 80% of drug is released in water in 90 minutes, and although there is some precipitation, the amount remaining in solution is substantial, even at 2 hours.

### Examples 10-18

Since the lactic acid containing formulation showed excellent dissolution rate, we have screened different carboxylic acids formulations of ziprasidone and the formulation compositions are presented in the table below.

| Example # | Ingredients | mg/Capsule |
|---|---|---|
| 10 | Ziprasidone | 80 mg |
| | Lactic acid | 90 mg |
| | Avicel 101 | 90 mg |
| | Aerosil | 30 mg |
| | Lactose | 90 mg |
| 11 | Ziprasidone | 80 mg |
| | Tartaric acid | 150 mg |
| | Aerosil | 100 mg |
| 12 | Ziprasidone | 80 mg |
| | Citric acid | 210 mg |
| | Aerosil | 100 mg |
| 13 | Ziprasidone | 80 mg |
| | Acetic acid | 60 mg |
| | Aerosil | 100 mg |
| 14 | Ziprasidone | 80 mg |
| | Lactic acid | 90 mg |
| | Aerosil | 50 mg |
| | Gelucire 44/14 | 20 mg |
| | Ac-Di Sol | 30 mg |
| | HPMC 3 CPS | 30 mg |
| 15 | Ziprasidone | 80 mg |
| | Lactic acid | 90 mg |
| | TPGS 1000 | 50 mg |
| | Ac Di Sol | 20 mg |
| | HPMC 3 CPS | 30 mg |
| | Aerosil | 30 mg |
| 16 | Ziprasidone | 80 mg |
| | Lactic acid | 90 mg |
| | Aerosil | 50 mg |
| | Gelucire 44/14 | 20 mg |
| | TPGS 1000 | 20 mg |
| | Ac-Di Sol | 30 mg |
| | HPMC 3 CPS | 30 mg |
| 17 | Ziprasidone | 80 mg |
| | Lactic acid | 90 mg |
| | Aerosil | 50 mg |
| | Gelucire 44/14 | 20 mg |
| | Ac-Di Sol | 30 mg |
| | HPMC 3 CPS | 30 mg |
| | SLS | 20 mg |
| 18 | Ziprasidone | 80 mg |
| | Lactic acid | 90 mg |
| | TPGS 1000 | 50 mg |
| | Ac Di Sol | 20 mg |
| | HPMC 3 CPS | 30 mg |
| | Aerosil | 30 mg |
| | SLS | 20 mg |

All these formulations along with Geodon capsules were tested in the following dissolution media:
Medium A: Plain DI water
Medium B: SGF (0.1 N Hydrochloric acid and 0.2% Sodium Chloride)
Medium C: Phosphate Buffer with pH 6.8
Medium D: FDA Published Method For Geodon Tier 1: Phosphate system with 2% SLS
Medium E: FDA Method Published Method for Geodon Tier 2: Phosphate system with Pancreatin

Mediums A, B and C are for dissolution evaluation, while the Mediums D and E are FDA published methods for 100% drug release which may not differentiate the excipients that are affecting the dissolution due to the presence of SLS and pancreatin. The dissolution profiles of different formulations in these media are summarized in the table below

| Example # | Sample | Medium | Medium | Medium | | |
|---|---|---|---|---|---|---|
| & dosage | timing | A | B | C | Medium D | Medium E |
| Geodon | 10 min | 5.5 | 3.2 | 11.7 | 54.8 | 30.2 |
| 80 mg | 20 min | 8.0 | 5.1 | 12.0 | 88 | 85 |
| | 30 min | 21.2 | 4.0 | 11.9 | 100.3 | 100 |
| | 45 min | 15.0 | 3.0 | 10.3 | 110 | 110 |
| | 60 min | 14.5 | 2.1 | 10.0 | 110 | 110 |
| Geodon | 10 min | 2.3 | 2.8 | 5.0 | Not Done | Not Done |
| 20 mg | 20 min | 2.4 | 5.1 | 7.5 | | |
| | 30 min | 3.5 | 2.1 | 7.8 | | |
| | 45 min | 5.5 | 1.8 | 7.8 | | |
| | 60 min | 1.8 | 1.9 | 10.4 | | |
| 09 | 10 min | | 5.2 | 12.0 | 62.0 | 30.0 |
| 80 mg | 20 min | 70.8 | 5.3 | 11.8 | 92.5 | 101.0 |
| | 30 min | | 5.5 | 12.1 | 105.5 | 109.0 |
| | 45 min | 57.0 | 6.2 | 11.8 | 110 | 110 |
| | 60 min | 55.1 | 7.2 | 10.5 | 110 | 110 |
| 09 | 10 min | 45.0 | 19.6 | 18.3 | 45.8 | 32.2 |
| 20 mg | 20 min | 55.3 | 22.5 | 20 | 86.3 | 75.9 |
| | 30 min | 65.4 | 24.3 | 21 | 98.8 | 97.2 |
| | 45 min | 42.1 | 25.0 | 21 | 105.6 | 105.4 |
| | 60 min | 45.1 | 24.6 | 22.5 | 105.1 | 104 |
| 10 | 10 min | | 3.8 | 11.1 | 52.5 | 42.3 |
| 80 mg | 20 min | 35.1 | 3.8 | 10.7 | 96.5 | 80.5 |
| | 30 min | | 4.4 | 11.2 | 107 | 98.5 |
| | 45 min | 44.3 | 5.8 | 12.2 | 111 | 109 |
| | 60 min | 56.4 | 4.9 | 11.2 | 109 | 106 |
| 11 | 10 min | 10.2 | 4 | 10.0 | 42.3 | 35.9 |
| 80 mg | 20 min | 9.8 | 3.3 | 10.2 | 86.5 | 78.9 |
| | 30 min | 19.6 | 3.8 | 9.8 | 103 | 100 |
| | 45 min | | | 10.2 | 113 | 106 |
| | 60 min | 10.3 | 4.1 | 9.9 | 115 | 110 |
| 12 | 10 min | 10.0 | 2.8 | 10.0 | 36.8 | 33 |
| 80 mg | 20 min | 10.0 | 3.5 | 10.2 | 84.6 | 76.3 |
| | 30 min | 22.6 | 3.2 | 9.8 | 98.9 | 101 |
| | 45 min | | | 10.2 | 106 | 102 |
| | 60 min | 9.3 | 3.8 | 9.9 | 112 | 100 |
| 13 | 10 min | 44.0 | Not | 9.0 | 36.2 | Not Done |
| 80 mg | 20 min | 1.0 | Done | 10.1 | 54.9 | |
| | 30 min | 1.4 | | 9.5 | 65.6 | |
| | 45 min | 1.5 | | 9.5 | 65.6 | |
| | 60 min | 0.6 | | 9.2 | 82.1 | |
| 14 | 10 min | 24.7 | 20.87 | 21.1 | 57.6 | 44.4 |
| 20 mg | 20 min | 30.0 | 24 | 25.3 | 70.0 | 89.2 |
| | 30 min | 30.3 | 24.7 | 26.2 | 87.0 | 102 |
| | 45 min | 28.7 | 25 | 26.5 | 100 | 105 |
| | 60 min | 28.7 | 26.3 | 26.5 | 110 | 105 |
| 15 | 10 min | 23.3 | 19.5 | 21.6 | 55.3 | 45.2 |
| 20 mg | 20 min | 35.1 | 22.1 | 23.5 | 88.6 | 89.3 |
| | 30 min | 30.0 | 22.5 | 22.2 | 104 | 105 |
| | 45 min | 25.5 | 26 | 28.6 | 106 | 106 |
| | 60 min | 26.0 | 26.2 | 29.5 | 106 | 101 |
| 16 | 10 min | 28.2 | 20.2 | 29.6 | 56 | 41.0 |
| 20 mg | 20 min | 29.9 | 23.9 | 25.1 | 84.2 | 86.6 |
| | 30 min | 30.6 | 26.6 | 23.1 | 96.6 | 101 |
| | 45 min | 26.9 | 26.0 | 23.6 | 102 | 106 |
| | 60 min | 27.4 | 22.3 | 24.2 | 108 | 101 |
| 17 | 10 min | 24.2 | 20.2 | 19.5 | 49.9 | 40.0 |
| 20 mg | 20 min | 28.0 | 26.5 | 29.5 | 76.5 | 82.6 |
| | 30 min | 26.5 | 22.4 | 26.3 | 92.3 | 97.6 |
| | 45 min | 25.5 | 20.6 | 22.5 | 102 | 104 |
| | 60 min | 26.8 | 18.5 | 22.0 | 102 | 105 |
| 18 | 10 min | Not | 22.2 | 21.8 | 39.5 | 34.6 |
| 20 mg | 20 min | Done | 23.3 | 25.0 | 77.0 | 71.1 |
| | 30 min | | 28.9 | 25.5 | 96.6 | 100 |
| | 45 min | | 28.5 | 26.5 | 106 | 101 |
| | 60 min | | 27.5 | 24.5 | 110 | 100 |

As shown in the Table above lactic acid containing formulations showing a better dissolution profiles in all tested mediums compare to the other formulations.

### Example 19

In addition to the above formulations, we have also made ziprasidone pellets by dissolving ziprasidone in an organic solvent or combination of organic solvents and coating on non-peril seeds.

### Composition:

| | |
|---|---|
| Ziprasidone | 20 mg |
| HPMC | 15 mg |
| PEG 20K | 10 mg |
| Non Pareil seeds | 85 mg |
| Solvet system: (3:2) | |
| Dichloromethane | |
| IPA | |

Manufacturing Procedure for Coated Pellets:

A Wurster chamber is fitted to a Fluidized Bed Processor and pre-heated for about 20 min. Non pareil seeds (NPS) of 16/20 size are loaded into the Wurster chamber for pre-warming under following conditions:
Inlet air temp . . . 38 deg C.
Bed Temp . . . 28 deg C.
Blower motor RPM . . . 1600
Time . . . 20 min
   In the interim, a ziprasidone dispersion was prepared by dissolving the HPMC 3 cps in Dichloromethane:IPA (60:40) mixture to get 1.5% w/w solution. PEG 20000 was added to the HPMC solution and stirred for about 5 min. Ziprasidone Hydrochloride was added to the solution under stirring and the dispersion was kept under stirring during the entire coating process. The dispersion was then coated onto the pre-warmed NPS under following conditions:
Inlet air temp . . . 38 deg C.
Bed Temp . . . 28 deg C.
Blower motor RPM . . . 1600-1800
Spray pump RPM . . . 2-5
Atomization pressure . . . 1.4-1.8 bar
Time . . . 20 min
   After completion of coating, the pellets were dried in FBP under mild conditions with drying time of about 10 min with following conditions:
Inlet air temp . . . 30 deg C.
Blower motor RPM . . . 1800
Atomization pressure . . . 1.4-1.8 bar
   These pellets were analyzed by HPLC and taken further for dissolution studies in five media by filling in the hard gelatin capsules. The dissolution data are presented in the table below:

| Examp # | Sample | Medium | Medium | Medium | | |
|---|---|---|---|---|---|---|
| & dosage | timing: | A | B | C | Medium D | Medium E |
| 19 20 mg | 10 min | 46.6 | 43.4 | 22.1 | 55.0 | 45.6 |
| | 20 min | 45.3 | 49.3 | 31.9 | 80.0 | 81.2 |
| | 30 min | 41.5 | 46.8 | 32.9 | 110 | 110 |
| | 45 min | 41.7 | 48.5 | 28.8 | 110 | 110 |
| | 60 min | 39.6 | 46.2 | 27.9 | 110 | 110 |

The most striking feature of this formulation is that it showed the best dissolution profile in SGF. In addition, the formulations of Examples 9-18 can also be coated on non-peril seeds. For example, the contents of the composition cited in example 16 without Aerosil and Ac-di-sol, can be dissolved in an organic solvent or a mixture of organic solvents and can be coated on non-peril seeds. Alternatively, using an extrusion process pellets of TPGS 1000 or Gelucire 44/14 or a combination of both and the drug by itself or in combination with other excipients dissolved in an organic solvent or combination of organic solvents and sprayed on the pellets prepared above.

I/we claim:

1. A method of enhancing the solubility of ziprasidone or a salt thereof comprising formulating at least
(a) at least one ziprasidone compound and at least an excipient component
(b) that includes at least one of
(i) one or more of a mono-, di-, or tri-ester of C₁₂₋₂₄fatty acids and glycerol, in which each fatty acid group is chosen independently of the others, or mixtures thereof; and/or
(ii) one or more mono- or di-esters of C12-24fatty acids and polyC2-3alkyleglycol, in which each fatty acid group is chosen independently of the others, or mixtures thereof; and/or
(iii) a vitamin E TPGS (Vitamin E tocopherol-succinic acid-polyethyleneglycol);
and where this component (b) may optionally include
(iv) optionally free polyC₂₋₃alkyleglycol;
(v) optionally free glycerol; and
(vi) optionally free fatty acids having 12-24 carbon atoms; and
(vii) mixtures thereof;
the formulation further comprising
(c) at least one surfactant selected from anionic and non-ionionic surfactants and still further comprising;
(d) at least one hydroxylalkyl alkylcellulose in which each alkyl group and each hydroxyalkyl group independently has from 1 to 4 carbon atoms.

2. The method of claim 1 wherein said component (b) is Gelucire or Vitamin E TPGS or mixtures thereof, said anionic surfactant is sodium lauryl sulfate, and said hydroxyalkyl-alkylcellulose is hydroxypropyl methylcellulose.

3. The method of claim 1 wherein said hydroxylalkyl alkylcellulose has a viscosity at 25° C. at a 2% concentration in water of up to about 50 cps.

4. The method of claim 2 said hydroxyalkyl-alkylcellulose is hydroxypropyl methylcellulose, 3 cps.

5. The method of claim 1 wherein said at least one ziprasidone compound is dispersed in or dissolved in a melt of said component (b) and along with said surfactant and said melt is formed into spheres and said spheres are coated with said hydroxylalkyl alkylcellulose.

6. The method of claim 1 wherein at least said ziprasidone compound and said component (b) are dissolved or dispersed in an organic solvent to form a solution or dispersion and said solution or dispersion is spray dried into spheres or pellets; said spheres or pellets then being blended with at least said surfactant and said hydroxylalkyl alkylcellulose.

7. The method of claim 1 wherein at least said ziprasidone compound and said component (b) are dissolved or dispersed in an organic solvent to form a solution or dispersion and said solution or dispersion is spray onto inert spheres or pellets; said coated spheres or pellets then being blended with at least said surfactant and said hydroxylalkyl alkylcellulose.

8. A pharmaceutical composition comprising at least
(a) at least one ziprasidone compound and
at least an excipient component (b) that includes at least one of
(i) one or more of a mono-, di-, or tri-ester of C₁₂₋₂₄fatty acids and glycerol, in which each fatty acid group is chosen independently of the others, or mixtures thereof; and/or
(ii) one or more mono- or di-esters of C12-24fatty acids and polyC2-3alkyleglycol, in which each fatty acid group is chosen independently of the others, or mixtures thereof; and/or
(iii) a vitamin E TPGS (Vitamin E tocopherol-succinic acid-polyethyleneglycol);
and where this component (b) may optionally include
(iv) optionally free polyC₂₋₃alkyleglycol;
(v) optionally free glycerol; and
(vi) optionally free fatty acids having 12-24 carbon atoms; and
(vii) mixtures thereof;
the formulation further comprising
(c) at least one surfactant selected from anionic and non-ionionic surfactants and still further comprising;
(d) at least one hydroxylalkyl alkylcellulose in which each alkyl group and each hydroxyalkyl group independently has from 1 to 4 carbon atoms.

9. The composition of claim 8 having
(a) a dissolution profile in water of not more than about 50% in 10 minutes, not less than about 25% and not more than about 70% in 20 minutes; not less than about 30 and not more than about 80% in 30 minutes, not less than about 30 and not more than about 80% in 45 minutes, and not less than about 30 and not more than about 80% in 60 minutes; and/or
(b) a dissolution profile in simulated gastric fluid (0.1N HCl in 0.2% NaCl aqueous solution) of not more than about 50% in 10 minutes, not less than about 25% and not more than about 70% in 20 minutes; not less than about 30 and not more than about 80% in 30 minutes, not less than about 30 and not more than about 80% in 45 minutes, and not less than about 30 and not more than about 80% in 60 minutes; and/or
(c) a dissolution profile in phosphate buffer at pH 6.8 of not more than about 50% in 10 minutes, not less than about 25% and not more than about 70% in 20 minutes; not less than about 30 and not more than about 80% in 30 minutes, not less than about 30 and not more than about 80% in 45 minutes, and not less than about 30 and not more than about 80% in 60 minutes; and/or
(d) about 20% to about 70% in 10 minutes and not less than about 80% in 30 minutes n the FDA Published Method for GEODON Tier 1 in phosphate system with 2% sodium lauryl sulfate; and/or
(e) about 20% to about 70% in 10 minutes and not less than about 80% in 30 minutes n the FDA Published Method for GEODON Tier 2 in phosphate system with pancreatin.

10. The composition of claim 9 meeting at least 2 of profiles (a)-(e).

11. The composition of claim 9 meeting all 5 of dissolution profiles (a)-(e).

12. A method of enhancing the bioavailability of ziprasidone over that exhibited by the marketed product GEODON comprising formulating at least
(a) at least one ziprasidone compound with
at least an excipient component (b) that includes at least one of
(i) one or more of a mono-, di-, or tri-ester of C₁₂₋₂₄fatty acids and glycerol, in which each fatty acid group is chosen independently of the others, or mixtures thereof; and/or
(ii) one or more mono- or di-esters of C12-24fatty acids and polyC2-3alkyleglycol, in which each fatty acid group is chosen independently of the others, or mixtures thereof; and/or
(iii) a vitamin E TPGS (Vitamin E tocopherol-succinic acid-polyethyleneglycol);
and where this component (b) may optionally include
(iv) optionally free polyC₂₋₃alkyleglycol;
(v) optionally free glycerol; and
(vi) optionally free fatty acids having 12-24 carbon atoms; and
(vii) mixtures thereof;
the formulation further comprising
(c) at least one surfactant selected from anionic and non-ionionic surfactants and still further comprising;
(d) at least one hydroxylalkyl alkylcellulose in which each alkyl group and each hydroxyalkyl group independently has from 1 to 4 carbon atoms.

13. A method of reducing the side effect profile seen with GEODON comprising administering a therapeutically equivalent but lower dose of ziprasidone than that in the therapeutically equivalent dosage form of GEODON comprising administering to a patient in need of ziprasidone therapy the pharmaceutical formulation of claim 7.

14. A method of treatment of a psychological condition selected from the group consisting of schizophrenia, bipolar mania and depression and/or metabolic disorders with ziprasidone or a salt thereof comprising administering said ziprasidone or pharmaceutically acceptable salt thereof to a patient in need of such treatment in the form of a composition of claim 7.

15. The composition of claim 7 wherein said composition is in the form of a generally spherical particle which is formed by granulation, trituration, extrusion, spray drying, or by coating an inert spheroidal substrate.

16. The composition of claim 7 further in unit dosage form comprising a swallow tablet, an oral disintegrating tablet, a capsule, a lozenge, a powder for dissolution, a lotion, an ointment, a cream, or a trandsdermal product.
